(19) United States
(12) Patent Application Publication
(10) Pub. No.: US 2006/0017575 A1
(43) Pub. Date: Jan. 26, 2006

(54) Alert system and method for an implantable medical device

(75) Inventor: Sean B. McAdams, Minneapolis, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 10/977,074

(22) Filed: Oct. 29, 2004

Related U.S. Application Data
(60) Provisional application No. 60/589,250, filed on Jul. 20, 2004.

(51) Int. Cl. G08B 23/00; A61B 5/00; A61B 5/05; A61B 5/02

(52) U.S. Cl. 340/573.1; 128/903; 600/547; 600/485; 600/365

(57) ABSTRACT

An alert system and method for providing alerts indicating an occurrence and a termination of an event relating to a patient. An implantable medical device, implanted within a patient, detects the occurrence of the event. Next an alert signal is wirelessly transmitted from the implantable medical device to provide an alert indicating the occurrence of the event. The implantable medical device then monitors for the termination of the event. When the termination of the event is detected, the implantable medical device wirelessly transmits an end alert signal to provide an alert indicating the termination of the event.

### CROSS-REFERENCE TO RELATED APPLICATION(S)

This application claims the benefit of U.S. Provisional Application No. 60/589,250 filed Jul. 20, 2004 for “Alert System And Method For An Implantable Medical Device” by J. Willenbring, J. VanDanacker, P. Krause, J. Masoud, J. Ball, H. Vitense, S. McAdams, and D. Hooper.

### INCORPORATION BY REFERENCE

U.S. Provisional Application No. 60/589,250 filed Jul. 20, 2004 for “Alert System And Method For An Implantable Medical Device” by J. Willenbring, J. VanDanacker, P. Krause, J. Masoud, J. Ball, H. Vitense, S. McAdams, and D. Hooper is hereby incorporated by reference in its entirety.

U.S. Non-Provisional application Ser. No. ______ (Atty. Dkt. P-20168.00) filed on even date for “Alert System And Method For An Implantable Medical Device” by J. Willenbring, J. VanDanacker, P. Krause, J. Masoud, J. Ball, H. Vitense, S. McAdams, and D. Hooper is hereby incorporated by reference in its entirety.

U.S. Non-Provisional application Ser. No. 10/727,008 filed Dec. 3, 2003 for “Method And Apparatus For Detecting Change In Intrathoracic Electrical Impedance” by Robert W. Stadler, et al. is hereby incorporated by reference in its entirety.

### BACKGROUND OF THE INVENTION

The present invention relates to implantable medical devices, and more particularly to an alert system for an implantable medical device.

During the latter portion of the twentieth century, it became common to implant medical devices to provide therapy for a vast number of medical conditions. Such devices included electrical stimulation devices, pain control devices, and drug delivery systems. Additionally and as these devices became more complex, it became necessary to monitor both their operation and the patient's condition.

At the same time, patients with implantable medical devices (IMDs) have come to expect a fuller life post-implant. These expectations often include few, if any, restrictions on their lifestyle. Thus, patients expect a great degree of mobility while their medical condition is being monitored and/or treated by the IMD and their physician. Semi-annual or annual in-office checkups for the IMD and the patient limits the frequency of monitoring. Moreover, the patient feels that he or she must remain close to the care giver's clinic or the hospital where checkups take place. Further, emergency situations may sometimes occur which, in the mind of the elderly patient, demand a very close proximity to the attending care giver. Going to the clinic for frequent check-ups may impose a considerable burden on the patient as well as an overall increase in the cost of healthcare. Accordingly, some IMDs are equipped with a communication system that connects to an interface in such a manner that it is transparent to the patient and yet provides the medical data required by the care giver.

Until recently, data transmission systems within IMDs were only capable of transferring data over a very small distance. Recent advances in wireless telemetry systems, often utilizing radio frequency (RF) systems, have opened the door to a whole host of new technologies. These technologies are reducing the burden on patients to perform routine tasks and are allowing patients to live with greater freedom and fewer restrictions on their lifestyle. However, there still exist multiple ways in which wireless telemetry systems can be utilized to further enhance the freedom of patients and the quality of care that they receive.

Patient interaction is often required in prior IMDs at the occurrence of an event. For example, an alarm system creates an audible alarm to alert the patient to the occurrence of an event. The patient then must either initiate a transfer of data to the care giver over the telephone or like systems or must immediately contact a care giver who can assess the situation. A patient who is relying on the therapy of an IMD can become very distressed when an audible alarm in the IMD begins to sound. Conversely, an audible alarm may not be heard by the patient, depending on the patient's hearing and environment.

### BRIEF SUMMARY OF THE INVENTION

The present invention relates to an alert system and method for informing and communicating to a preceptor (e.g. patient; care giver; or interested party) the occurrence and termination of an event related to a patient. The system generally includes an implantable medical device implanted within a patient. Specifically, the implantable medical device includes means for detecting the occurrence and termination of the event, and a means for transmitting an alert signal and an end alert signal.

### BRIEF DESCRIPTION OF THE DRAWINGS

FIG. 1 illustrates an embodiment of the alert system of the present invention.

FIG. 2 is a block diagram of an IMD of the alert system of the present invention.

FIG. 3 is a block diagram of a monitor of the alert system of the present invention.

FIG. 4 is a block diagram of a patient management network of the alert system of the present invention.

FIG. 5 is a block diagram of patient management web clients of the alert system of the present invention.

FIG. 6 is an exemplary flow diagram illustrating a method of sending an alert signal from the IMD upon the occurrence of an event satisfying alert criteria.

FIGS. 7 and 8 are exemplary screen shots of a user interface allowing the care giver to select clinical management alert settings.

FIGS. 9 and 10 are exemplary screen shots of a user interface allowing the care giver to select lead/device integrity alert settings.

FIG. 11 is an exemplary flow diagram illustrating a method of sending an alert signal when an event occurs and an all-clear signal when an event terminates.

FIG. 12 is a more detailed embodiment utilizing an alert signal and an end alert signal.

FIG. 13 shows an alternate communications system by which alert signals may be transmitted to a medical support network.

### DETAILED DESCRIPTION

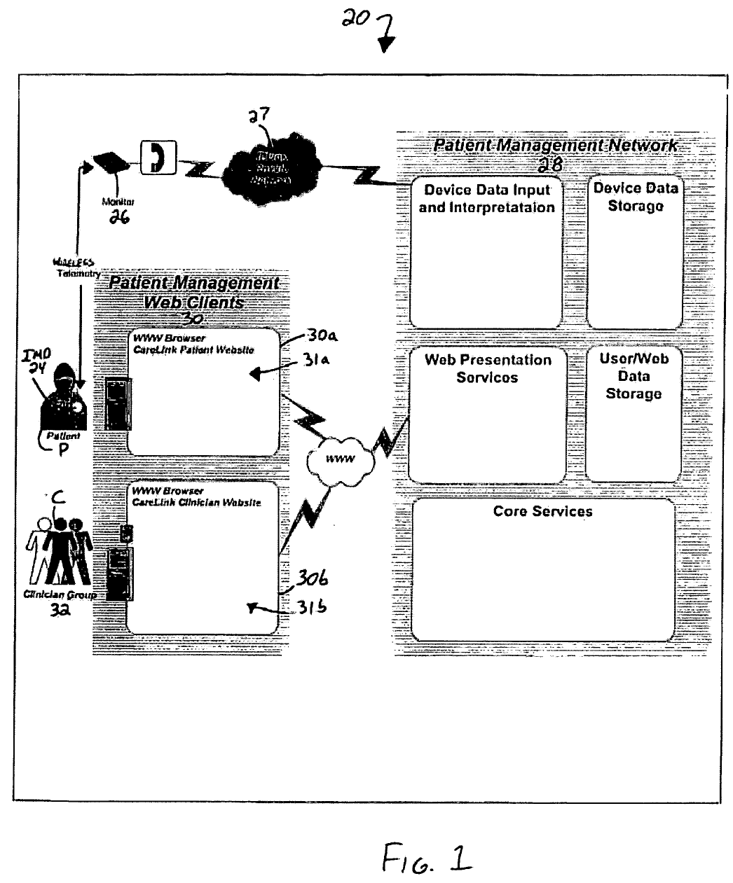

FIG. 1 illustrates an embodiment of alert system 20 of the present invention, which communicates between patient P and care giver C. Alert system 20 includes implantable medical device (“IMD”) 24 within patient P, monitor 26, private network 27, patient management network 28, and patient management web clients 30 including patient browser 30a that is capable of displaying patient website 31a and care giver browser 30b that is capable of displaying care giver website 31b.

IMD 24 is, for example, a device such as a pacemaker or defibrillator that is implanted within patient P and is capable of providing cardiac therapies. These therapies may include providing pacing pulses or defibrillation shocks to the heart of patient P. IMD 24 also records useful data such as, for example, without limitation, data related to the condition of patient P, therapy delivery, device performance and functionality and periodically provides information to care giver C. IMD 24 also provides self-monitoring of the system operation (such as lead impedance data, high-voltage capacitor charge times, battery capacity, etc.). In addition, IMD 24 is capable of detecting the occurrence of an event that satisfies predefined alert criteria. The alert criteria may pertain to a clinically-relevant event or a result of self-diagnosis of the IMD that may be necessary, for example, to inform the patient or an interested person. Once an event has been detected that satisfies an alert criterion, IMD 24 is capable of providing a perceptible alert. A patient alert is a patient notification of a triggered alert criteria via an audible tone vibration, or other perceptible communication directed to the patient from IMD 24 or monitor 26. A silent alert is a notification of a triggered alert criteria via alert system 20, which is not perceptible to the patient.

Monitor 26 is an instrument, such as Medtronic's CareLink monitor, intended for use in a patient's home that is capable of receiving data from the patient's implanted device via telemetry and transmitting this information via phone lines or other communication platforms to private network 27 which transfers the data to patient management network 28. Private network 27 is, for example, the IP Link service from MCI, which provides a private, secure, and reliable connection.

Patient management network 28 utilizes secure computer servers that collect, process and store data sent from monitor 26. This information is available to patient P and care giver C through e.g., patient management web clients 30. Patient management web clients 30 are computer systems with a browser capable of viewing web pages on the World Wide Web. At least two patient management web clients 30 are provided: a patient browser 30a and a care giver browser 30b. Patient P can access data and other information on patient website 31a via patient browser 30a. Care giver C can access data and other information on care giver website 31b via care giver browser 30b.

There are three follow-up scenarios in which care giver C can interact with IMD 24 to monitor the condition of patient P and IMD 24: standard follow-up, remote follow-up, and ambulatory follow-up. Standard follow-up is a scheduled face-to-face interaction between patient P and care giver C in order to check the patient's health and the functioning of IMD 24. Typically, the standard follow-up occurs every three to six months. Alert system 20 of the present invention reduces the number of standard follow-ups that need to take place. The remote follow-up is a scheduled electronic transmission of the data stored in IMD 24 to care giver C in order to check the health of patient P and the functioning of the patient's IMD 24. Similar to the standard follow-up, the remote follow-up typically occurs every three to six months depending upon the patient's medical condition. The remote follow-up is enabled by use of monitor 26 and patient management network 28. The ambulatory follow-up is an unscheduled and IMD-initiated electronic transmission of the data stored in IMD 24 to care giver C in order to alert care giver C to the occurrence of an event that satisfies the alert criteria and allow care giver C to check the health of patient P and the functioning of the patient's IMD 24. It has been found that standard follow-ups are time consuming, and inconvenient for both patient P and care giver C. Ambulatory follow-ups, however, can be provided by alert system 20 of the present invention to provide many benefits.

Communication between the various components of alert system 20 will now be described. Either upon the detection of an event satisfying an alert criterion, or at a scheduled time, IMD 24 is interrogated by monitor 26 over a wireless telemetry system utilizing radio frequency (RF) signals. This interrogation provides data from IMD 24 to monitor 26. Monitor 26 communicates the data to patient management network 28 over a standard telephone system from the home of patient P and through private network 27. Data is then displayed to care giver C or patient P using patient management web clients 30 utilizing the standard world-wide web (“WWW”) secured communication protocol (e.g. SSL).

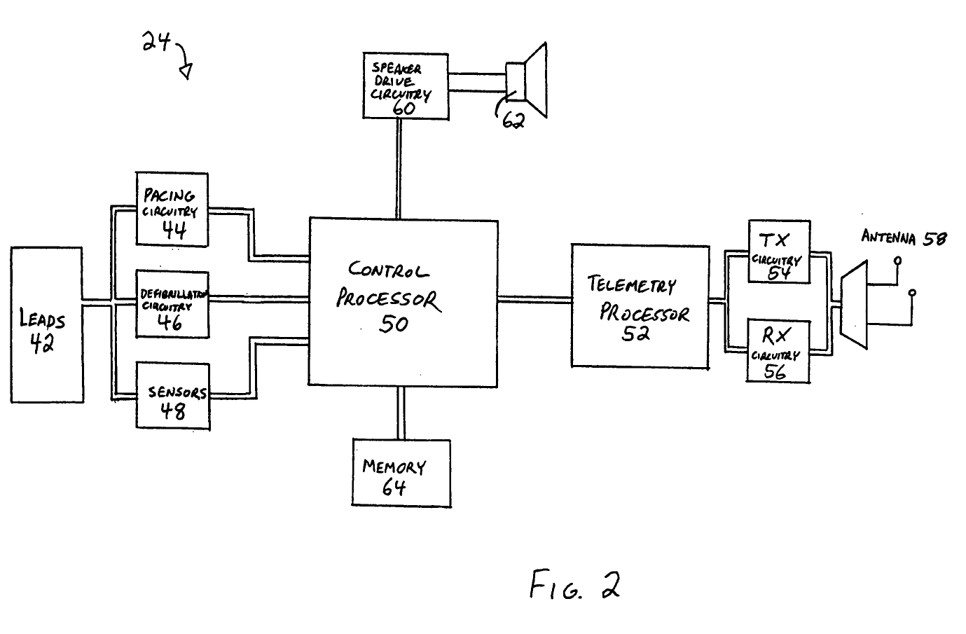

FIG. 2 is a block diagram of IMD 24 of alert system 20. Although it is recognized that alert system 20 can be used with any type of implantable medical device, a specific example will now be provided in which IMD 24 is an implantable cardioverter defibrillator. IMD 24 includes leads 42, pacing circuitry 44, defibrillation circuitry 46, sensors 48, control processor 50, telemetry processor 52, transmitter circuitry 54, receiver circuitry 56, antenna 58, speaker drive circuitry 60, speaker 62, and memory 64. Control processor 50 is the primary controller for IMD 24 and thus control processor 50 controls the overall operation of IMD 24.

Control processor 50 controls pacing circuitry 44 and defibrillation circuitry 46 to provide therapeutic electrical pulses to leads 42. Leads 42 are preferably implanted within the heart of patient P and provide an electrically conductive path for the pulses to selected locations within the heart. In addition, leads 42 can be used by sensors 48 to detect cardiac signals in the heart. These cardiac signals are conducted through leads 42, detected by sensors 48, and then provided to control processor 50. If desired, control processor 50 can save the signals in memory 64, which is preferably a type of random access memory (RAM) or flash memory.

Control processor 50 is capable of analyzing the cardiac signals received from sensors 48 and determining whether an event has occurred which satisfies an alert criterion. In addition, control processor 50 is capable of monitoring the condition of IMD 24 to determine whether an event has occurred which satisfies an alert criterion. If control processor 50 determines that such an event has occurred, it then decides, based upon care giver selectable alert settings, what type of an alert should be provided. If the care giver selectable alert settings instruct control processor 50 to provide a patient alert, an alert signal is generated and sent to speaker drive circuitry 60. Speaker drive circuitry 60 provides the necessary electrical signal to speaker 62 to create an audible sound which alerts patient P to the occurrence of the event.

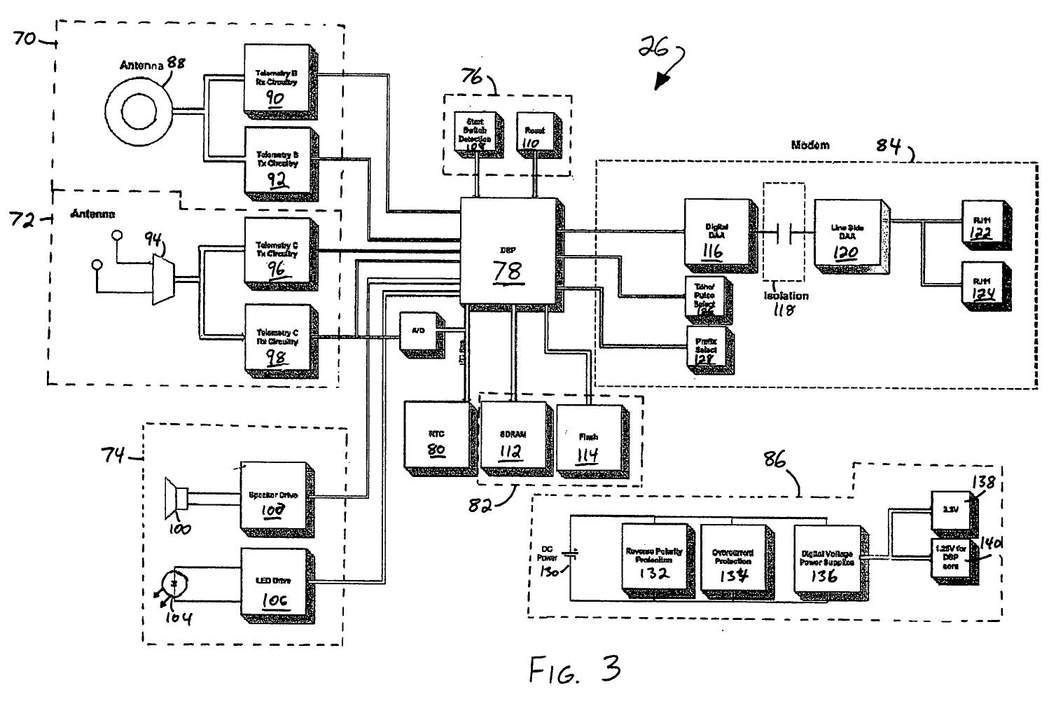

Alternatively, if care giver selectable alert settings instruct control processor 50 to provide a silent alert, then control processor 50 instructs telemetry processor 52 to transmit a wireless telemetry signal. Telemetry processor 52 then controls transmitter circuitry 54 to create a radio frequency (RF) signal that is transmitted wirelessly over antenna 58. This signal alerts monitor 26 (FIG. 3) that IMD 24 is initiating communication, provided that monitor 26 is within the telemetry range of IMD 24. In this way, IMD 24 is capable of initiating communication with monitor 26 to inform monitor 26 of the occurrence of an event that satisfies the alert criteria. Further detail of the communication between IMD 24 and monitor 26 will be provided with reference to FIG. 3.

IMD 24 could be utilized to provide an alert signal in response to any detectable event as well as the absence of any detectable event. In one embodiment there are six types of events, the occurrence of which care giver C may choose to be notified of. The six types of events relate to therapy delivery, arrhythmias, heart failure, system integrity, cardiac ischemia, and edema.

Therapy delivery events occur when IMD 24 provides a therapy to patient P. These therapies may include electrical stimulation, defibrillation, drug delivery, or the delivery of other agents. The alert criterion may specify, for example, that an alert should be sent for all therapies, an initial therapy, an unsuccessful therapy, a successful therapy, an attempt at providing a therapy, a delayed or aborted therapy, only after a predetermined number of therapies, a defibrillation therapy that was not able to be delivered due to an unsuccessful attempt to charge its delivery capacitor, or a defibrillation therapy that was not successfully delivered due to a short circuit present in its delivery pathway. In addition, the alert criterion may specify, for example, that an alert should be sent for a change in percentage of pacing, a change in rate responsive therapy, a change in use of therapies, or a newly detected need for a therapy.

Arrhythmic events may include, for example, a new atrial or ventricular tachycardia, a new atrial or ventricular fibrillation, a non-sustained tachycardia, an atrioventricular nodal reentry tachycardia, a premature ventricular contraction, a detected episode with no therapy programmed, a change in duration of episodes, a frequency of episodes, a rate of arrhythmia, and a presence of rapid atrial conduction to ventricle, or when there is no intrinsic rhythm detected (asystole).

Heart failure events may include edema triggers, pressure data triggers (for example, data from an implantable hemodynamic monitor such as the Medtronic Chronicle® device), heart rate variability, activity, and nocturnal heart rate changes. Edema, for example, has been found to be a strong indicator of heart failure, and can be used to detect heart failure before any other symptoms are detectable by the patient. Intra-thoracic impedance relates the volume of fluid between a pair of electrodes and thus pulmonary edema (e.g., measuring the impedance between an electrode disposed within a chamber or major vessel (e.g., superior vena cava) of a heart and an surface electrode disposed in a housing, or “can,” of IMD 24 or the conductive surface of the can itself. This impedance measurement can be used to detect the amount of fluid in the lungs, and the onset of pulmonary edema. The reader should note that while the term “transthoracic impedance” is used within this document, it should be interpreted as “intra-thoracic” or “inter-thoracic” impedance (e.g., impedance measured between in-dwelling electrodes disposed in or about the thoracic cavity of a subject). The impedance measurements may occur among one or more subcutaneous electrodes, endocardial electrodes, epicardial electrodes, pericardial electrodes and the like. Furthermore, while the generic term “edema” is oftentimes employed in this document, the term should be interpreted as relating more to pulmonary edema than to peripheral edema, although the onset of one form of edema can precipitate onset or increase in severity of the other form of edema.

System integrity events are events which indicate an abnormal functioning of IMD 24. System integrity events may include a memory failure (such as with RAM), a power-on reset (POR), a charge circuit timeout, an elective replacement indicator (ERI), device hardware failure, EEPROM failure, device initialization failure, multiple microprocessor failure, capture threshold changes, sensing threshold changes, presence of far-field R wave oversensing, myopotentials, electromechanical interference (EMI), T-wave oversensing, pacemaker modes of VOO or DOO on for more than a predetermined amount of time, device detection off or therapy off, device has not had telemetry session in a predetermined amount of time, no superior vena cava lead when active can is off, and an excessive number of non-physiologic ventricular or atrial intervals.

An ischemia event is a deficiency of blood in tissue, usually due to functional constriction or actual obstruction of a blood vessel. An example of an ischemia event is cardiac/myocardial ischemia which is a deficiency of blood supply to areas of heart tissue.

FIG. 3 is a block diagram of monitor 26 of alert system 20 of the present invention. Monitor 26 includes short-range (e.g., programming head) communication system 70, longer-range wireless communication system 72, patient alerts 74, control switches 76, digital signal processor (“DSP”) 78, real-time clock (“RTC”) 80, memory 82, modem 84, and power supply 86. Short-distance communication system 70 includes antenna 88, receiver circuitry 90, and transmitter circuitry 92. Wireless communication system 72 includes wireless antenna 94, wireless transmitter circuitry 96, and wireless receiver circuitry 98. Patient alerts include speaker 100, speaker drive 102, light-emitting diodes (LEDs) 104, and LED drive 106. Control switches 76 include start switch 108, and reset 110. Memory 82 includes SDRAM 112 and flash 114. Modem 84 includes digital data access arrangement (“digital DAA”) 116, isolation 118, line side DAA 120, RJ11 ports 122 and 124, tone/pulse select 126, and prefix select 128. Power supply 86 includes DC power 130, reverse polarity protection 132, overcurrent protection 134, digital voltage power supplies 136, and DC outputs 138 and 140.

Monitor 26 is located within the home or in the vicinity of where patient P is present. In addition, multiple monitors could be located at different places to allow communications with IMD 24 through any one of the monitors. Monitor 26 is capable of longer-distance wireless communication with IMD 24 via wireless communication system 72. Short-distance communication system 70 is also provided to enable communication with implantable medical devices which utilize the short-distance head-based communication systems.

Antenna 88 of short-distance communication system 70 is preferably a dual opposing coil RF read head which is used to transmit data during downlinks and receive data during uplinks. Short-distance receiver circuitry 90 amplifies, filters, and digitizes the received data signal before sending it to DSP 78. Short-distance transmitter circuitry 92 receives logic level signals from DSP 78 and converts them to a higher current drive signal for RF read head antenna 88. Transmitter circuitry 92 properly tunes the antenna to the appropriate frequency for transmission. Short-distance transmitter circuitry 92 is also capable of being disabled to isolate it from antenna 88 so that it does not affect the receive circuitry during receive mode.

Wireless communication system 72 provides the capability of communicating with IMD 24 using wireless telemetry with RF signals. Wireless antenna 94 includes two separate antennas to provide spatial diversity. It is tuned to a nominal wireless telemetry carrier frequency with sufficient bandwidth to accommodate the entire medical implant communication service (“MICS”) 402-405 MHz band. Wireless transmitter circuitry 96 generates the RF downlink transmission to IMD 24 in the 403-405 MHz MICS band. Wireless receiver circuitry 98 receives and demodulates the RF uplink transmission to IMD 24 in the 402-405 MHz MICS band.

Patient alerts 74 include speaker 100 coupled to speaker drive 102 and LEDs 104 coupled to LED drive 106. Speaker drive and LED drive are both controlled by DSP 78. Speaker drive 102 and speaker 100 serve two functions: to generate tones to indicate an error or alert condition, and to make modem 84 audible. Speaker drive 102 multiplexes an audible tone from DSP 78 and the modem audio. Speaker drive 102 also has the ability to take a logic level signal as an input and drive the speaker at a high enough current to meet audio sound pressure level requirements. LEDs 104 are used as visual indicators to give status indications to patient P or (care giver C) during an interrogation and modem connection. LEDs 104 also alert patient P to power status and completion of uploaded data to the server. Light from LEDs 104 is transferred to a user interface overlay via injection molded optical light pipes. LED drive 106 accepts a logic signal from DSP 78 or digital DM 116 and drives LEDs 104 at a higher current. Switches 76 provide buttons which allow patient P to interact with monitor 26. Switches 76 include start switch detection 108 and reset 110. Start switch 108 allows patient P to instruct monitor 26 to begin an interrogation of IMD 24. Reset 110 allows patient P to reset monitor 26 to factory defined settings.

DSP 78 is responsible for the majority of the functions of monitor 26. It encodes and transmits data for both short-distance and wireless downlink transmissions, and decodes digitized data from the corresponding receiver circuitry 90 or 98 during uplink transmissions. DSP 78 is also used to implement a soft modem and directly interfaces with digital DAA 116 to send data out on a phone line. DSP 78 also runs the TCP/IP, PPP, and HTTP client software on top of the modem software. All user interface functions are handled by DSP 78 including control of patient alerts 74, as well as reading the status of tone/pulse select switch 126 and prefix select switch 128.

Real-time clock 80 is provided in monitor 26 to keep track of the time. Both IMD 24 and monitor 26 keep track of the time so that communication can take place at predetermined times. In order to save battery power in IMD 24, the telemetry system of IMD 24 does not remain active at all times. Instead, IMD 24 and monitor 26 have predefined communication times during which routine communication can take place. However, as described above, alert system 20 also includes the capability of IMD 24 initiated communication at any time in which an event is detected which satisfies the alert criteria.

Memory 82 includes SDRAM 112 and flash 114. SDRAM 112 is used to store interrogation data from IMD 24 as well as program code and other program-related data. Flash 114 is used to store program data and any parameters that need to be stored in non-volatile memory (e.g. phone numbers). DSP 78 boots from flash 114.

Modem 84 includes digital DAA 116, isolation 118, line side DAA 120, RJ-11 jacks 122 and 124, tone/pulse select switch 126, and prefix select switch 128. Digital DAA 116 along with DSP 78 and line side interface 120 form a complete V.34 modem. As described above, DSP 78 is used to implement a soft modem and directly interfaces with digital DAA 116 to send data out on a phone line. DSP 78 also runs the TCP/IP, PPP, and HTTP client software on top of the modem software. DSP 78 also reads the status of tone/pulse select switch 126 and prefix select switch 128. Tone/pulse select switch allows patient P to select whether dialing modem 84 should use tone or pulse dialing. Prefix select switch 128 allows patient P to select whether a prefix needs to be dialed to access an outside line, such as a number 9. Digital DAA 116 interfaces with DSP 78 through a serial interface and contains all of the control registers for modem 84 such as termination settings, clock phase-locked loop (“PLL”) settings, etc. Digital DAA 116 also includes an audio output (not shown) that is coupled to speaker drive 102 that multiplexes the modem audio and the tones generated from DSP 78. Line side DM 120 is connected directly to the phone line via RJ-11 jacks 122 and 124. Line side DAA 120 generates DTMF signals that allow it to communicate over a telephone system to private network 27, as well as providing several other necessary functions (overload protection, programmable terminations to generate an off-hook condition for various countries, 2 to 4 wire conversion, etc.). Modem 84 isolates the line side from the other components of monitor 26 through capacitive isolation barrier 118.

Power supply 86 provides DC power to monitor 26. Power supply 86 includes DC power source 130, reverse polarity protection 132, overcurrent protection 134, digital voltage power supplies 136, and DC outputs 138 and 140. The function of power supply 86 should be easily understood by one skilled in the art and therefore will not be described in further detail.

Monitor 26 is a portable interrogation and data transfer tool used with IMD 24. Monitor 26 offers the capabilities to patient P, care giver C, and service personnel of remote interrogations, data processing, reporting and follow-up to be performed when the patient is at home and the care giver is in the clinic or a location that has web-enabled capability. This remote feature allows for reduced travel and waiting time, providing prompt care to patients and better efficiencies to care givers. It also enables care givers to better manage patients and still maintain the quality of care that is warranted in the marketplace. Furthermore, monitor 26 allows field representatives to increase their productivity, provide equal or better service to existing and new customers worldwide, and control costs for providing the services. The increased productivity is obtained by reducing the time required for manufacturer-assisted follow-up. Monitor 26 performs four primary functions: it interrogates IMD 24 and stores the data, it collaborates with patient management network 28 to confirm the establishment of a connection with patient management network 28, it performs any required file translation functions necessary for data transfer, and it executes the data file transfer and then collaborates with patient management network 28 to confirm that the data file transfer was successful. Although the preferred embodiment of the present invention utilizes monitor 26, it is recognized that other devices could also be used to perform the function of monitor 26. Examples of such devices include a telemetry transponder/repeater, a cell phone, or a Bluetooth-enabled or WiFi-enabled communication device.

Now that the structure of IMD 24 and monitor 26 have been described, the communications between IMD 24 and monitor 26 will be described. As explained above, care giver C and IMD 24 interact for standard follow-up, remote follow-up, and ambulatory follow-up. Of these, a remote follow-up and an ambulatory follow-up utilize monitor 26 as one of the communication links between IMD 24 and care giver C. An ambulatory follow-up occurs only when IMD 24 detects the occurrence of an event that satisfies the alert criteria and must be communicated to care giver C. A remote follow-up, on the other hand, is scheduled and expected by both IMD 24 and monitor 26, and therefore is initiated by monitor 26. This procedure also satisfies current FCC regulations for implantable medical device operating in the MICS band to initiate communications only if a “medical implant event” occurs. (Title 47 of the Code of Federal Regulations, Part 95.628.) The FCC has further defined the event as an occurrence that necessitates data exchange in order to maintain patient safety.

In any event, once communication has been established, monitor 26 performs an interrogation of IMD 24. Control processor 50 of IMD 24 reads the desired data from memory 64 and then provides it to telemetry processor 52. Telemetry processor 52 and transmitter circuitry 54 transform the data to an RF signal that is wirelessly transmitted by antenna 58 to monitor 26. Monitor 26 receives the wireless transmission of data through antenna 94 and wireless receiver circuitry 98. Receiver circuitry 98 then provides the data to DSP 78 which stores the data in SDRAM 112. After all desired data has been received, the communication between monitor 26 and IMD 24 is closed.

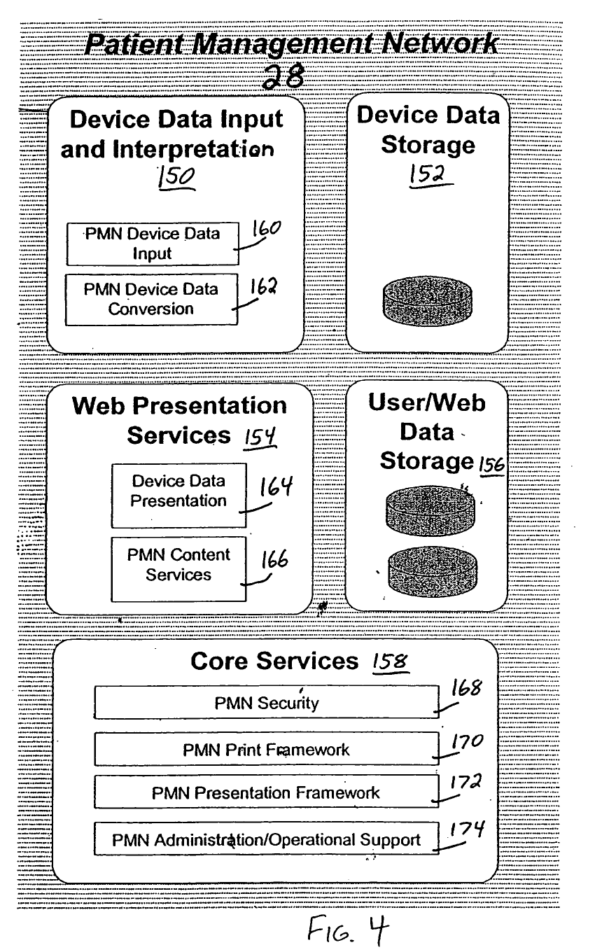

FIG. 4 is a block diagram of patient management network 28 of alert system 20 of the present invention. Patient management network 28 (“PMN”) includes device data input and interpretation 150, device data storage 152, web presentation services 154, user/web data storage 156, and core services 158. Device data input and interpretation 150 includes PMN device data input 160 and PMN device data conversion 162. Web presentation services 154 include device data presentation 164 and PMN content services 166. Core services 158 include PMN security 168, PMN print framework 170, PMN presentation framework 172, and PMN administration/operational support 174.

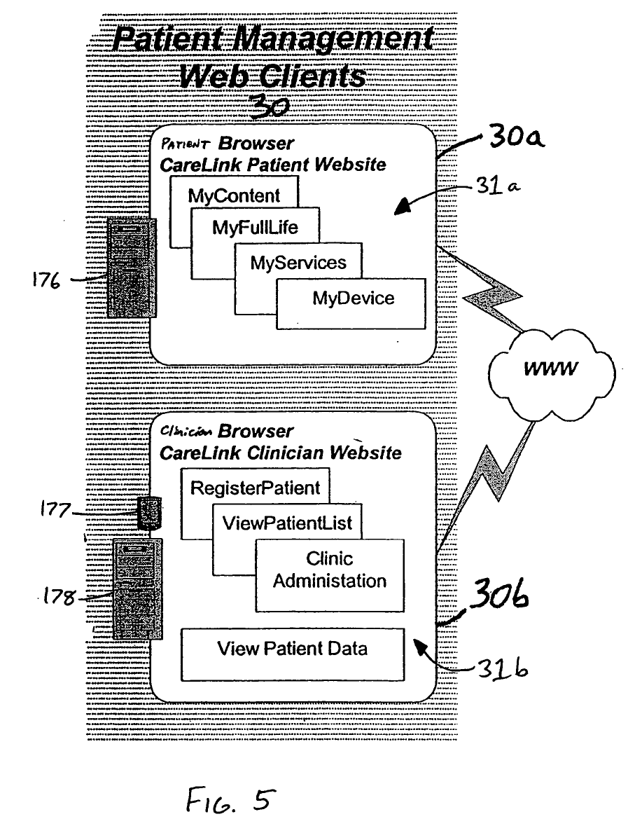

Patient management network 28 utilizes a series of secure computer servers that collect, process and store data sent from monitor 26. This data is then made available to patient P and care giver C through Internet accessible websites that are personalized for their particular needs. The patient and care giver websites will be described in further detail with reference to FIG. 5.

After monitor 26 has completed a full interrogation of IMD 24, it then transfers the data over a telephone line to private network 27. One example of private network 27 is MCI's IP Link private network. Private network 27 allows monitor 26 to remotely access patient management network 28 over a private, secure, and reliable connection utilizing the hypertext transfer protocol (“HTTP”). Patient management network, which consists of a series of secure computer servers, receives the data from monitor 26 (over the private network) and into device data input and interpretation 150, and more specifically through PMN device data input 160 which preferably includes a dedicated router. The data is then processed by PMN device data conversion 162 and stored in device data storage 152. For example, further processing is performed by web presentation services 154 to turn the raw device data into viewable portable document format (“PDF”) documents, graphs, tables, etc. and also to create client and patient personalized websites which are accessed by patient browser 30a and care giver browser 30b. This data is then stored in user/web data storage 156. Additionally, core services 158 are performed by patient management system 28 to provide PMN security 168, PMN framework 170, PMN presentation framework 172, and PMN administration/operational support 174.

FIG. 5 is a block diagram of patient management web clients 30 of alert system 20 of the present invention. Patient management web clients 30 include patient computer 176 running patient browser 30a, and care giver's personal digital assistant (“PDA”) 177 or care giver computer 178 both capable of running care giver browser 30b. Patient browser 30a is used by patient P to access the patient website 31a from patient management network 28 through the world-wide web. Care giver browser 30b is used by care giver C to access the care giver website 31b.

Patient and care giver websites 31a and 31b are both generated by patient management network 28. Patient website 31a includes: general information modules (not related to the patient's IMD data) concerning the patient's device and their disease; general (“wire feed”) news items or articles containing information on medical topics of interest; psychosocial support modules designed to meet the needs of specific patient groups; access to a personalized “storefront” of products designed to meet the patient's needs; a virtual on-line community of “friends and family” that can share information and experiences with the patient; and views of IMD 24 data supplied from the device data level. Care giver website 31b includes the following capabilities: creation and maintenance of a patient list with various features for customization on a patient-specific basis; customized updates on products, clinical trials and research in addition to the provision of general (“wire-feed”) news items or articles containing information on medical topics of interest; a route for care givers to access technical services; views of stored IMD data and alerts supplied from the device data level; and means for posting information on the site that their patients can read.

Now that the structure of alert system 20 has been described, further detail will be provided as to the operation of alert system 20 of the present invention.

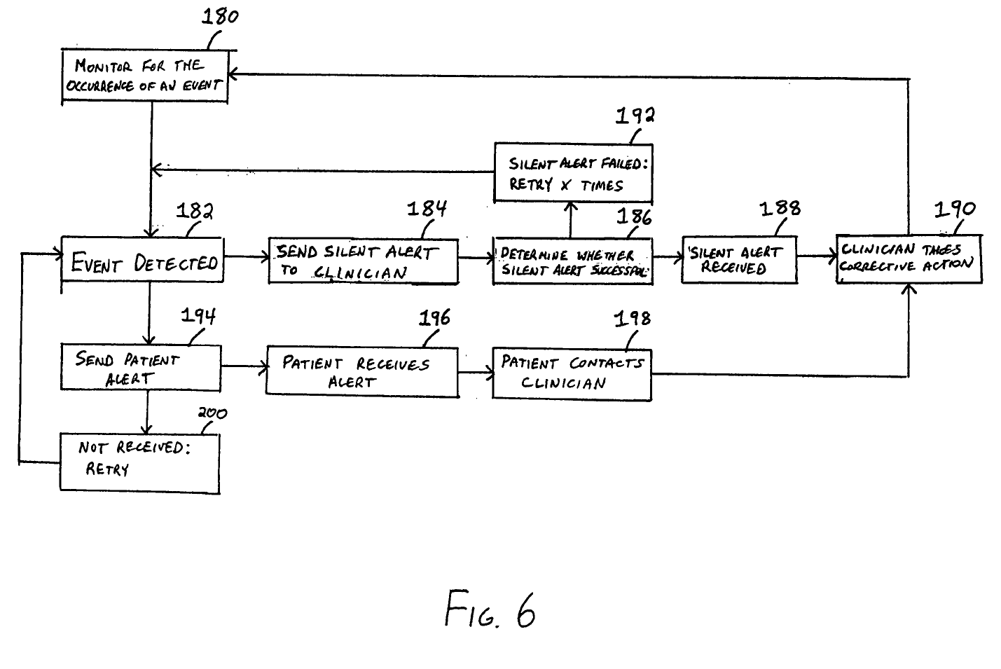

FIG. 6 is an exemplary flow diagram illustrating a method of sending an alert signal from IMD 24 upon the occurrence of an event satisfying the alert criteria. The method is intended only as an exemplary embodiment. IMD 24 begins by monitoring for the occurrence of an event (step 180). Once an event is detected (step 182), the system decides who should first be notified of the occurrence of the event based upon predefined alert criteria. This step is preferably performed by IMD 24, but may also be performed by monitor 26 or patient management network 28. If IMD 24 decides to attempt a silent alert to care giver C (step 184), IMD 24 wirelessly transmits an alert signal to monitor 26. If monitor 26 receives the alert signal, monitor 26 performs a full interrogation of IMD 24, as defined above, and closes the session. Monitor 26 then transfers the data to patient management network 28, which informs the care giver of the occurrence of the event. The system then determines whether the silent alert was successfully communicated to the care giver (step 186).

Various methods of determining the success of the silent alert may be used. For example, monitor 26 can provide a verification signal to IMD 24 after monitor 26 has successfully transferred the data to patient management network 28, care giver C can provide a verification signal to patient management network 28 which is then sent through alert system 20 to IMD 24, or success can be defined as a successful transfer of data from IMD 24 to monitor 26 which would require no verification signal. If alert system 20 determines that the silent alert has been received (step 188), it knows that care giver C will take the necessary corrective action (step 190). If alert system 20 determines that the silent alert has failed (step 192) (for example, if no verification signal is received in a predetermined amount of time), then IMD 24 assumes that the alert was not successfully communicated to care giver 30. As a result, IMD 24 repeats the attempted transmission a predetermined number of times (steps 182, 184, 186, and 192). Since the most frequent cause of a failed transmission is that IMD 24 is not in range of monitor 26, it is preferable to wait for a specified amount of time, such as three hours, before retrying the transmission. For example, IMD 24 will continue attempting communication every three hours for up to three days for a total of twenty four times.

If repeated attempts to transmit the alert signal are unsuccessful, IMD 24 will then switch to the backup alarm. In the exemplary embodiment, the backup alarm is the patient alert that includes speaker drive 60 and speaker 62. Thus, after repeated unsuccessful attempts to wirelessly transmit the alert signal (steps 182, 184, 186, and 192), an alert signal is sent to the patient alert (step 194). Once the patient has received the alert signal (196), patient P contacts care giver C (step 198) to inform him or her of the occurrence of an event. In an exemplary embodiment, alert system 20 will continue to provide the patient alert periodically until alert system 20 verifies that the patient alert has been received. To do so, alert system 20 detects when a full interrogation of IMD 24 has been taken place, and recognizes at that point that the patient alert has been received and that care giver C will take the appropriate corrective action (step 190).

In alternate embodiments, the patient alert may also be triggered by other situations in which the wireless transmission is considered a failure, such as: when the alert signal is not received by monitor 26, when the interrogation of IMD 24 by monitor 26 does not complete, when patient management network 28 does not receive the interrogated data, when the care giver does not acknowledge an alert after being informed by patient management network 28, or when the care giver does not log in to care giver website 31b via care giver browser 30b and check the patient's data after being alerted by patient management network 28.

The present invention inherently includes software control (i.e., instructions performed by at least one computer processor) and, as applicable, the methods herein that are susceptible of being stored on a computer readable medium or being sent as control signals to affect a technical result are expressly disclosed and claimed herein.

Alert system 20 of the present invention provides a user interface in which care giver C can set the care giver selectable alert settings of alert system 20 to perform as desired. These settings define the alert criteria that are used by IMD 24 (or patient management network 28) to determine whether or not an alert should be sent, and whether a silent alert of a patient alert should be sent. Thus, alert system 20 provides care giver C with a user interface in which he or she can select which events should initiate a silent alert, a patient alert, both alerts, or no alert at all. Table 1 is an exemplary list of care giver selectable alert conditions. It includes the alert name, a description of the alert, and the programmable condition parameters available for that alert.

TABLE 1
Care Giver Selectable Alert Conditions

| | | PROGRAMMABLE |
|---|---|---|
| ALERT | | CONDITION |
| NAME | DESCRIPTION | PARAMETERS |
| Lead | A measured lead impedance | Independently |
| Impedance | trend value has exceeded the | enabled for each |
| Out of | acceptable range set for the | lead: If enabled, |
| Range | lead. | Minimum and |
| | | Maximum |
| | | Impedances. |
| Low | The elective-replacement- | Enable/disable |
| Battery | indicator (ERI) battery voltage | |
| Voltage | condition occurs for three | |
| | consecutive days, excluding | |
| | days when high voltage | |
| | charges took place. | |
| Excessive | Charging performance of | Enable/disable |
| Charge | device has met ERI indicator | |
| Time | for charge time. | |
| VT/VF | A ventricular tachyarrhythmia | Enable/disable |
| Therapies | occurred which required | |
| Exhausted | delivery of all enabled | |
| | therapies for the zone and | |
| | failed to terminate the | |
| | arrhythmia. | |
| Number of | The programmable number of | Enable/Disable |
| Ventric- | shocks, or more, were | and Number of |
| ular | delivered for a single VT/VF | Shocks |
| CV or | episode. | |
| Defibril- | | |
| lation | | |
| Shocks | | |
| VF | The device is not in session | Enable (High |
| Therapy | and six hours have elapsed | Urgency Only)/ |
| Disabled | since the last programming | Disable |
| | and one or more of the | |
| | following conditions still exist: | |
| | VF detection has been | |
| | disabled, or more than two VF | |
| | Therapies have been disabled, | |
| | or FVT is enabled to ‘via VF’ | |
| | and more than two FVT | |
| | therapies have been disabled. | |
| High | The measured threshold for | Independently |
| Threshold | the chamber is at 5 V for 1 day. | Enabled for each |
| | | paced chamber |
| AT/AF | The cumulative time that the | Enable/Disable: |
| Burden | patient has been in AT/AF in a | Time in AT/AF |
| | given day (since midnight) has | Threshold |
| | exceeded the acceptable | |
| | duration as set by the care | |
| | giver. | |
| Fast V | The patient has a mean | Enable/Disable: |
| Rate | ventricular rate while in AT/AF | Ventricular Rate |
| during | that has exceeded the | while in AT/AF |
| AT/AF | acceptable rate threshold, and | Threshold and |
| | AT/AF has occurred for a | Min. Time in |
| | minimum, cumulative duration | AT/AF Threshold |
| | as selected by the care giver | |
| | (may be a different duration | |
| | than AT/AF Burden duration). | |
| | Determined on a per day | |
| | basis. | |
| Thoracic | The fluid index exceeded the | Enable/Disable, |
| Fluid | threshold, indicating possible | Threshold, Alert |
| Overload | thoracic fluid accumulation in | Time |
| Alert | the patient. | |

Alert system 20 not only allows care giver C to enable or disable the alert conditions, but also allows care giver C to select the response to each condition. If care giver C selects the alert mode to be “audible,” the alert method is set as a patient alert. If care giver C selects the alert mode to be “silent,” the alert method will be a silent alert. Finally, if the user selects the alert mode to be “audible+silent,” both methods of notification will be used.

Additionally, a number of abnormalities always produce a notification and cannot be disabled by care giver C. These relate to catastrophic conditions requiring immediate follow-up and are shown in Table 2.

TABLE 2
Non-Programmable Alert Conditions

| | | PROGRAMMABLE |
|---|---|---|
| | | CONDITION |
| ALERT NAME | DESCRIPTION | PARAMETERS |
| Power-On | A POR has occurred | No selectable |
| Reset (POR) | | parameters |
| CPU Lockout | The device has entered the | No selectable |
| | CPU Lockout state | parameters |
| Charge Time- | An attempt to charge the high | No selectable |
| out | voltage therapy capacitors | parameters |
| | has aborted due to a time-out | |
| Incorrectly | The case electrode is | No selectable |
| Configured | disabled as a high voltage | parameters |
| Defibrillation | therapy electrode and there is | |
| System | no acceptable impedance for | |
| | a defibrillation pathway | |
| Permanent | The programmed pacing | No selectable |
| Asynchronous | mode was asynchronous at | parameters |
| Mode | midnight of the device clock, | |
| | and still asynchronous at the | |
| | alert alarm time | |

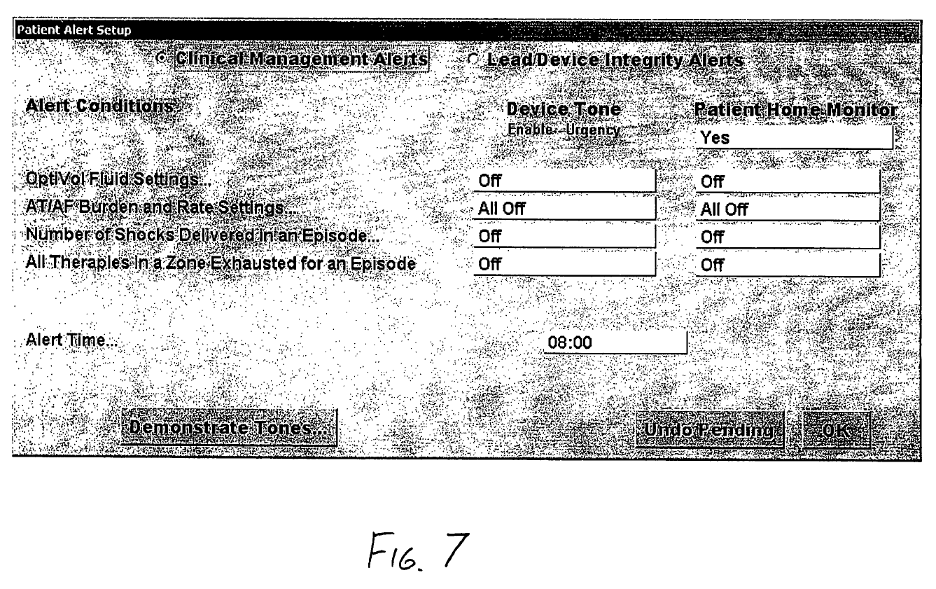

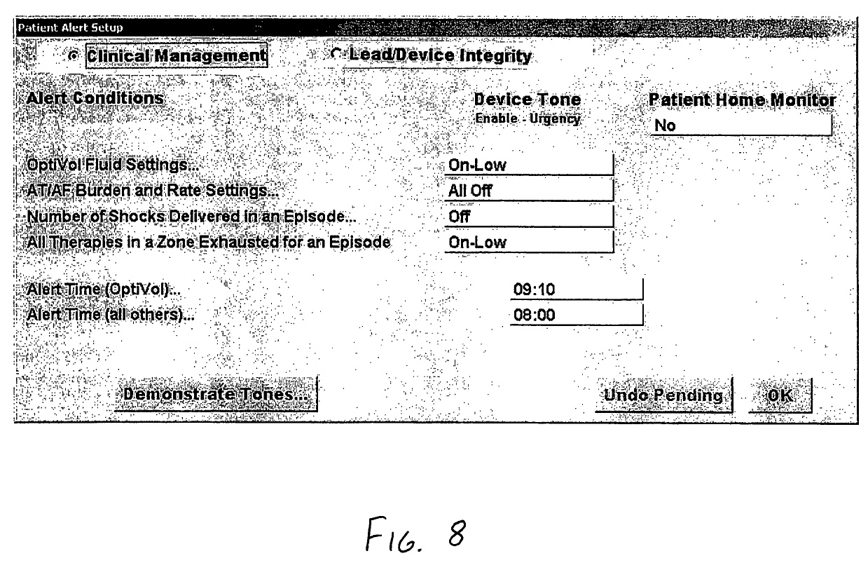

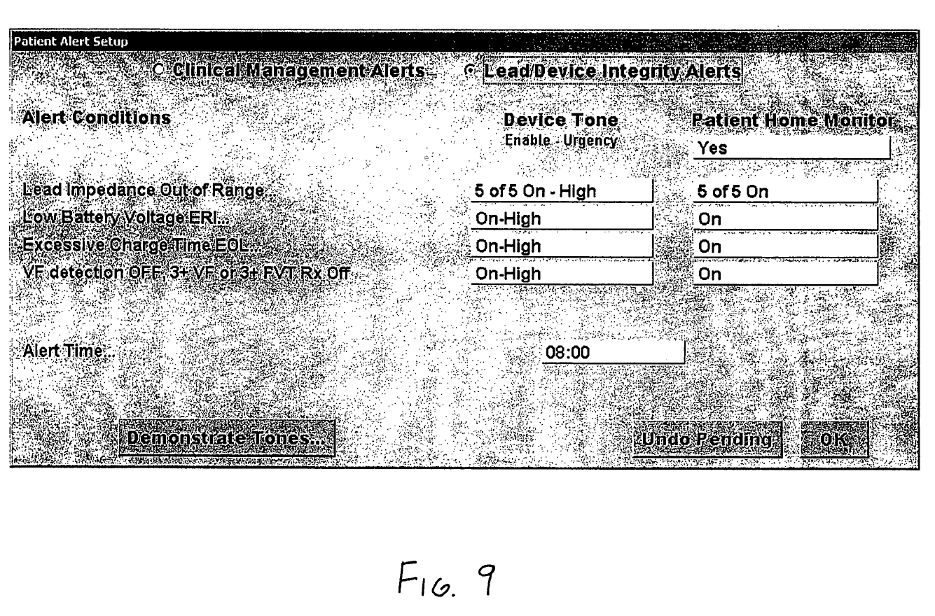

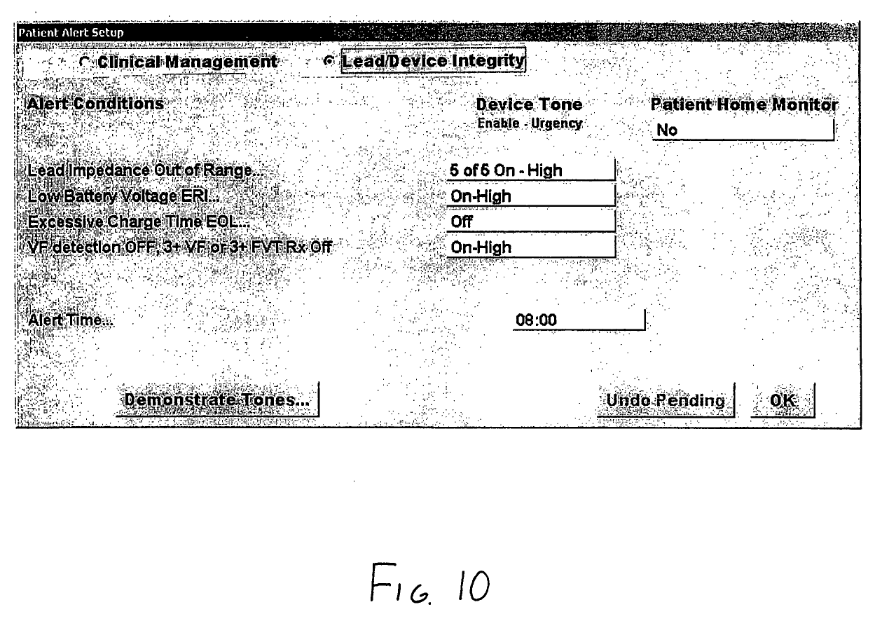

FIGS. 7-10 are exemplary screen shots of user interface 210 allowing care giver C to select care giver selectable alert settings. FIGS. 7 and 8 show clinical management alert settings and FIGS. 9 and 10 show lead/device integrity alert settings. FIG. 7 shows the dual-column clinical management alert settings that are available when “Patient Home Monitor” is set to “Yes” (enabled). FIG. 8 shows the single-column clinical management alert settings that are available when “Patient Home Monitor” is set to “No” (disabled). Similarly, FIGS. 9 and 10 show the lead/device integrity alert settings that are available when “Patient Home Monitor” is set to “Yes” or “No”. User interface 210 provides a plurality of menus and sub-menus through which care giver C can select the desired alert settings. User interface 210 may be provided to care giver C in a number of different embodiments. In a first exemplary embodiment, user interface 210 is provided on a programmer for IMD 24. A programmer for an implantable medical device is well known in the art and typically includes a computer-like system having a display and input devices such as a keyboard. The programmer also includes a communication device such as an RF head or a wireless telemetry system which allows the programmer to program IMD 24. In this embodiment, user interface 210 is displayed on the display of the programmer, such that care giver C is able to select the desired alert settings. After care giver C has selected the desired settings, the programmer programs the settings into IMD 24.

In a second exemplary embodiment, user interface 210 is provided by patient management system 28. In this embodiment, user interface 210 is displayed to care giver C as a part of care giver website 31b. Care giver C is able to select the desired alert settings through care giver website 31b and then save them to patient management network 28. Patient management network 28 then initiates communication through private network 27, and monitor 26 to IMD 24 where the care giver selectable alert settings are stored in IMD 24.

To select the desired settings, care giver C first selects the type of settings that he or she wishes to set: Clinical Management Alerts (FIGS. 7 and 8) or Lead/Device Integrity Alerts (FIGS. 9 and 10). Clinical Management Alerts relate to events involving the condition of patient P, while Lead/Device Integrity Alerts relate to events involving the condition of IMD 24 and attached leads.

The user interface provides the option of enabling or disabling care giver-selectable settings for the interaction between IMD 24 and monitor 26 altogether. This feature accommodates those patients that do not have a monitor. Care giver C selects whether monitor 26 should be enabled or disabled by selecting the “Patient Home Monitor” field and selecting “Yes” or “No.” If care giver C selects “Yes” then care giver-selectable silent alert options are enabled (FIGS. 7 and 9). If care giver C selects “No” then all silent alert options are disabled (FIGS. 8 and 10).

The desired settings are then selected from the menu and sub-menus as desired. For example, if care giver C wants to be alerted to an atrial tachycardia within patient P that exceeds a certain duration or exceeds a certain rate, care giver C would select the option that reads “AT/AF Burden and Rate Settings . . . ” Care giver C would then be provided with a sub-menu in which he or she could select the type of alert desired, the urgency of the alert, and the duration or heart rate at which the alert would trigger. In addition, care giver C is also able to select the specific time of the day in which a patient alert (“device tone”) is provided by selecting “Alert Time . . . ” Alert settings for Lead/Device Integrity Alerts are similarly chosen through the menus as shown in FIGS. 9 and 10 and additional sub-menus.

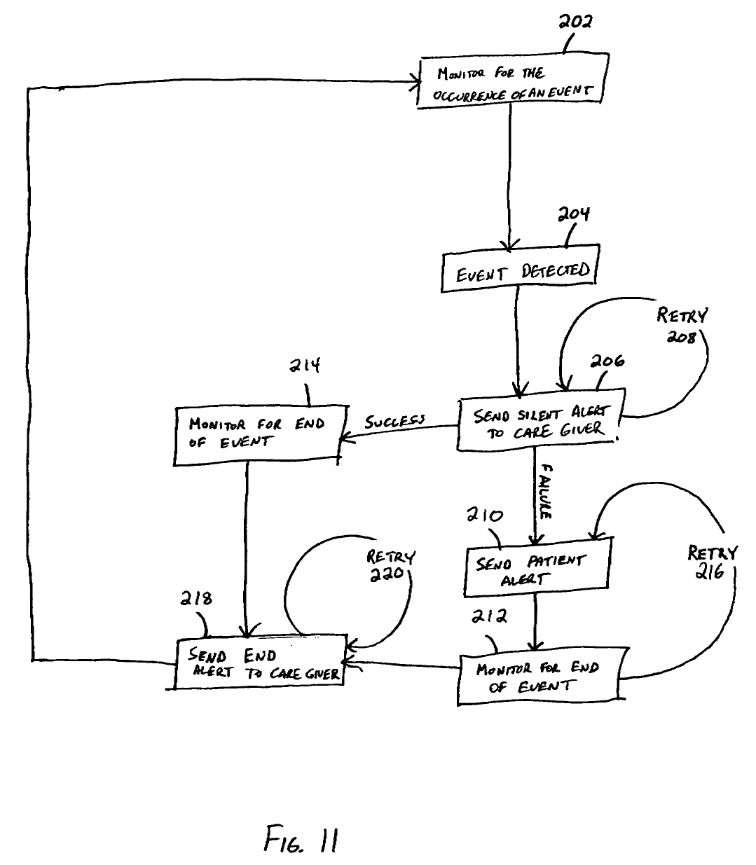

FIG. 11 is an exemplary flow diagram illustrating a method of sending an alert signal when an event occurs and an end alert signal when an event terminates. This method of providing alert signals allows a care giver to be notified not only of when an event begins, but also when that same event comes to an end.

The process of detecting an event and sending an alert signal remains the same as that previously described. IMD 24 monitors for the occurrence of an event (step 202). Once an event is detected (step 204), IMD 24 transmits a silent alert to the care giver (step 206). If the silent alert is unsuccessful, IMD 24 retries the transmission a predetermined number of tries (step 208). If the silent alert is still unsuccessful, IMD 24 activates the patient alert (step 210).

At this point, either the silent alert has been sent to care giver C, or the patient alert has been provided to alert patient P. IMD 24 then continues monitoring this event to detect the end of the event (steps 212 or 214). Meanwhile, if a patient alert was provided (step 210), this alert can be repeated periodically (step 216) to ensure that patient P has become aware of the occurrence of the event. When the end of the event is detected, IMD 24 transmits an end alert signal (step 218) in the same way that the alert signal was previously transmitted. If the transmission of the end alert signal is unsuccessful, IMD 24 retries the transmission (step 220). When the end alert signal is successfully transmitted, care giver C is notified that the event has terminated, in the same way that care giver C was notified of the occurrence of the event.

In an embodiment of the present invention, sensors 48 of IMD 24 (shown in FIG. 2) include a transthoracic impedance sensor. The transthoracic impedance sensor is used to measure the impedance between a lead in a chamber of the heart and the can of IMD 24. This impedance measurement can be used to detect the amount of fluid in the lungs, and the onset of edema. Edema has been found to be a strong indicator of heart failure, and can be used to detect heart failure before any other symptoms are detectable by the patient. Once edema has been detected, care giver C may choose to make use of one of a number of possible therapies. These therapies may include electrical stimulation from IMD 24 or medications.

The onset of edema can be detected by measuring the transthoracic impedance and comparing this to a threshold value or a normal range of values. The threshold value can be selected by a user, or set dynamically by IMD 24. The dynamic settings are provided by monitoring the patient over a period of time and determining the normal fluctuations in transthoracic impedance. The normal range, or a minimum threshold value is then calculated.

In any event, IMD 24 continues to monitor the transthoracic impedance, to determine whether the therapy is successful. If the therapy is successful, IMD 24 will detect a decrease in the transthoracic impedance, which indicates that the amount of fluid in the lungs has been reduced. Once the impedance measurement is reduced below a predetermined threshold value, IMD 24 provides end alert signal to the physician to inform the physician that the therapy has been successful.

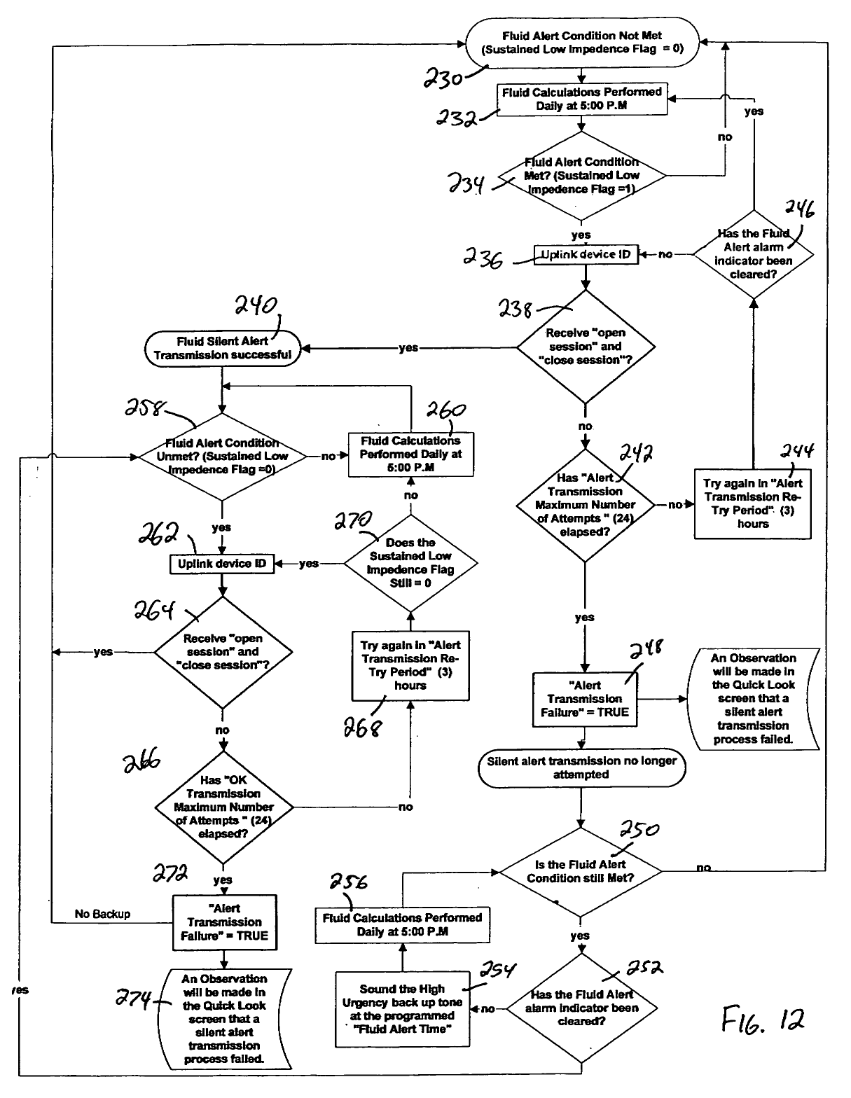

FIG. 12 shows a more detailed embodiment utilizing an alert signal and an end alert signal. This embodiment is described with reference to a transthoracic impedance sensor to detect the onset and termination of edema. However, it is recognized that the embodiment is equally useful and applicable for sensing any other detectable event. For example, care givers will likely find the present invention particularly useful for monitoring events in which the time between onset and termination of the event is a relatively long period of time, such as one day or more. Detectable events such as reduced transthoracic impedance, out of range blood pressure, glucose level excursions, neurological events, empty reservoir in a drug infusion device, out of range electrolytes or protein levels are all examples of detectable events in which a physician will not only want to know when the event occurs, but also when the event terminates, often as a result of drugs or other therapies, or interaction from the patient or a care giver. It is also recognized that mathematical calculations may be performed on any, or a combination of, sensed levels or events, the result of these calculations being compared to a threshold value or range of values to determine whether an event has occurred or terminated.

The method begins with the fluid alert condition not met (step 230). At this point a sustained low impedance flag in IMD 24 is set to “0” to indicate that the transthoracic impedance is above a threshold value. Fluid calculations are then performed periodically (step 232). For example, the fluid calculations may be performed daily at a predetermined time such as 5:00 p.m. Alternatively, in order to account for daily fluctuations in transthoracic impedance these measurements may be taken periodically throughout the day, for example twenty measurements may be spread out through the day between a period of noon until 5:00 p.m.

At the end of the period the measurements are compared to a threshold value set by care giver C. If the transthoracic impedance is below the predetermined threshold value the sustained low impedance flag is set to “1” to indicate that the fluid alert condition has been met (step 234). If the transthoracic impedance is not below the predetermined threshold value the sustained low impedance flag stays set at “0” and IMD 24 continues to monitor the patient for future occurrences. If the fluid alert condition has been met (step 234), IMD 24 then begins the process of sending a silent alert. This process begins with IMD 24 uplinking its device identification code (ID) to monitor 26 (step 236). The device ID may also include a medical event uplink code to indicate to monitor 26 that a medical event has occurred. The device ID informs monitor 26 that IMD wishes to communicate with it. Alternatively, a communication session can be scheduled in advance such that monitor 26 initiates the communication between IMD 24 and monitor 26. In any event, monitor 26 performs a full interrogation of IMD 24 by transmitting an open session command, receiving device data including data about the occurrence of the event, and then transmitting a close session command (step 238). If both the open session and close session commands are received by IMD 24, IMD 24 recognizes that the silent alert transmission was successful (step 240).

In another embodiment of the invention, IMD 24 includes a successful session bit. The successful session bit is turned ON (set to “1”) after monitor 26 successfully transmits the alert. When monitor 26 successfully transmits the alert, monitor 26 initiates communication with IMD 24 to program the successful session bit ON to inform IMD 24 that the alert has been successfully transmitted.

If IMD 24 did not receive both the open session and close session commands (step 238) (or if the successful session bit in IMD 24 has not been programmed ON by monitor 26), IMD 24 begins the silent alert retry process (steps 242-246 , and 236-238) in which IMD 24 retries transmission periodically (for example every three hours) for maximum number of attempts (such as twenty-four) or a maximum amount of time (such as three days). If the retry process is unsuccessful for a predetermined maximum number of attempts (step 242) IMD 24 recognizes that the silent alert transmission failed (step 248). As a result, IMD 24 stores an observation note that will inform care giver C, upon the next follow-up, that a silent alert was attempted and was unsuccessful.

IMD 24 then provides a patient alert (steps 250-256) to inform patient P that the fluid alert condition has been met. At this point IMD 24 verifies that the fluid alert condition is still met (step 250) and verifies that the fluid alert alarm indicator has not been cleared (step 252). If the fluid alert condition is not still met (step 250) IMD 24 returns to monitoring patient P for a future event (step 230). If IMD 24 determines that the fluid alert condition has still been met (step 250) and that the fluid alert alarm indicator has not been cleared (step 252), it then provides a patient alert such as a high urgency backup tone (step 254). This alert may be provided immediately or at the programmed “fluid alert time.” IMD 24 then continues with the patient alert retry process (steps 250-256). The retry process will repeatedly perform fluid calculations (step 256), determine whether the fluid alert condition is still met, whether the fluid alert alarm indicator has been cleared, and provide the patient alert if necessary.

After providing either a silent alert (step 240) or a patient alert (steps 250-256), IMD 24 begins monitoring for the end of the event. To do so, IMD 24 determines whether the fluid alert condition is unmet (in other words whether the sustained low impedance flag has returned to “0”) (step 258). If the event has not terminated, IMD 24 continues to monitor for the termination of the event by performing fluid calculations (step 260) and determining whether the fluid alert condition has been unmet (step 258). When the termination of the event is detected (such that the sustained low impedance flag is set to “0”) IMD 24 begins the process of sending a silent end alert signal.

To transmit a silent end alert signal, IMD 24 uplinks its device ID (step 262) to monitor 26. Monitor 26 then recognizes that IMD 24 wishes to communicate with it and performs a full interrogation of IMD 24. Alternatively, a communication session can be scheduled in advance, such that monitor 24 initiates the communication between IMD 24 and monitor 26. In any event, monitor 26 transmits an open session command, receives the interrogation data including data about the end of the event, and transmits a close session command (step 264). If IMD 24 receives both the open session and close session commands (or if the successful session bit has been programmed ON), IMD 24 recognizes that the silent end alert signal was successfully received and IMD 24 returns to monitoring for the occurrence of the next event (step 230). If both the open session and close session commands are not received by IMD 24 (step 264) (or the successful session bit has not been programmed ON), IMD 24 again begins a retry process (steps 262-270) in which the transmission is retried periodically (such as every three hours) for a maximum number of attempts (such as twenty-four) or a maximum amount of time (such as three days). If the retry process (steps 262-270) is unsuccessful, such that the silent end alert is not transmitted successfully, IMD 24 recognizes that the transmission of the silent end alert was not successful (step 272). IMD 24 then makes an observation note for the care giver, such that a caregiver will be informed upon the next follow-up that the silent end alert transmission was attempted but was not successful (step 272). IMD 24 then returns to monitoring for the occurrence of the next event (step 230).

In an embodiment of the invention, monitor 26 includes an LED to provide a visual indicator to patient P of the occurrence of an event. The LED is turned on when monitor 26 receives the silent alert. After the event terminates, and monitor 26 receives the silent end alert signal, the LED is turned off to indicate that the event has terminated.

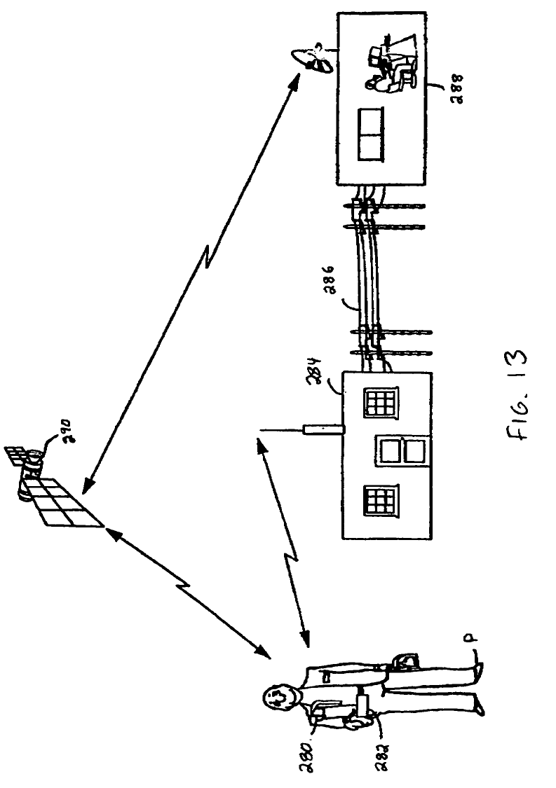

FIG. 13 shows an alternate communications system by which alert signals may be transmitted to a medical support network. IMD 280 is shown as being an implantable medical device, but could alternately be external rather than implantable. External device 282 is portable and carried with patient P and communicates with IMD 280 by wireless signals. External device 282 communicates with communications link transceiver 284 by wireless signals. Transceiver 284, in turn, communicates with medical support network 288 via telephone lines 286. Any of a number of forms of communication of data may be used. External device 282 may also communicate with medical support network 288 via communications link satellite 290. Once an alert is received by medical support network 288, care giver C is notified.

Although alert system 20 of the present invention has been described with reference to preferred embodiments, workers skilled in the art will recognize that changes may be made in form and detail without departing from the spirit and scope of the invention. The inventors have contemplated many changes which will be readily understood by one skilled in the art. For example, the alert system includes a number of patient alert methods including a speaker in IMD 24 and a speaker and LED's in monitor 26. Other known types of patient alerts may also be used including muscle stimulation, vibration, or olfactory stimulation (in an external device such as monitor 26). Furthermore, multiple patient alerts may be provided such that care giver C can select the most desired alert for the particular event. Similarly, various means of alerting the care giver (or any other person) are contemplated. An alert may be provided to a device worn or nearby a care giver such as a telemetry enabled watch, home PC, public access transponder, WiFi/Bluetooth network, telephone, pager, cell phone, or displayed on a programmer during the next interrogation. Alternatively, the alert could be provided to a call center from monitor 26 or patient management network 28, the call center having an operator who would contact the care giver. The alerts may include all information from the interrogation of IMD 24, or it may be simply a message informing care giver C to check care giver website 31b. Furthermore, a silent alert may be provided to alert patient P of the occurrence of an event in the same way that a silent alert is provided to alert care giver C of the occurrence of an event.

The exemplary embodiments of the invention store the care giver-selectable alert settings in memory 64 of IMD 24. It is recognized that the alert settings may also be stored in other locations such that other methods may be utilized. For example, care giver-selectable alert settings can be stored only on patient management network 28. In this embodiment, IMD 24 would send an alert signal to monitor 26 upon the occurrence of any possible event. Monitor 26 would then transfer the alert to patient management network 28. Patient management network 28 would then inform care giver C of the event only if the settings for that particular alert had been programmed ON. In another exemplary embodiment, the care giver-selectable alert settings are again stored on patient management network 28. IMD 24 then provides an alert signal frequently (preferably more than one per day) and the patient management network 28 would inform care giver C only if an event were detected which matched an event that was programmed ON in the care giver-selectable alert settings. Although these embodiments reduce the amount of memory needed on IMD 24 (or increase the amount of memory available for other data storage), they also decrease the longevity of the battery of IMD 24 by requiring more frequent transmission of data.

Furthermore, the alert system 20 is capable of providing an alert when an event is detected by IMD 24. It is recognized that this alert may be provided for any event which can be detected by IMD 24, or any other device in the system, or a system in communication with any device in the system. The event may include an event internal to IMD 24 , within patient P, or external to patient P. Sensors capable of detecting the event may include any known sensor such as cardiac sensors, blood sensors, neurological sensors, a global positioning system receiver, microphones, magnetic sensors, pressure sensors, temperature sensors, impact sensors, electric or magnetic field sensors, vibration sensors, chemical sensors, light sensors, radiation sensors, etc. In addition, control processor 50 can be utilized to perform calculations, check for patterns, or otherwise process data and provide an alert based upon a predetermined criterion. Thus, it should be understood that the alert system of the present invention provides enormous possibilities for improving the safety of patients, increasing the quality of care that they receive, and increasing their quality of life.

1. An improved method of providing clinician- and/or patient-alerts indicating a termination of an event relating to a patient, wherein a prior detected occurrence of an event by an implantable medical device (IMD) wirelessly transmits an alert signal from the IMD indicating the occurrence of the event, said improved method comprising:
detecting the termination of a previously detected event with an IMD; and
wirelessly transmitting an end-alert signal from the IMD indicating the termination of the event.

2. A method according to claim 1, further comprising:
receiving the end-alert signal with a monitor;
transferring the end-alert signal from the monitor to a patient management network; and
communicating information regarding the event that occurred and terminated to at least one of a care giver, a hospital, a clinic, a clinician, the patient, said information comprising at least one of:
an event duration, an event type, a time the event started, a time the event ended, an aggregate event burden, a device intervention code, an activity sensor input for the patient.

3. A method according to claim 1, further comprising:
receiving the end-alert signal with a monitor;
transferring the end-alert signal from the monitor to a patient management network; and
communicating to at least one of a care giver, a hospital, a clinic, a clinician, the patient, that the event has terminated.

4. A method according to claim 1, wherein detecting the occurrence of the event comprises determining when a measured intra-thoracic impedance is less than a threshold value.

5. A method according to claim 4, wherein detecting the termination of the event comprises determining when a measured intra-thoracic impedance returns to a value greater than the threshold value.

6. A method according to claim 1, wherein detecting the occurrence of the event and detecting the termination of the event comprise periodically performing a physiologic measurement.

7. A method according to claim 6, wherein periodically performing the measurement comprises one of performing a measurement at a predetermined time and a predetermined date.

8. A method according to claim 7, wherein the measurement comprises an intra-thoracic impedance measurement.

9. A method according to claim 6, wherein periodically performing the measurement comprises performing a series of measurements during a predetermined range of times each day.

10. A method according to claim 9, wherein the measurement comprises an intra-thoracic impedance measurement.

11. A method according to claim 1, wherein wirelessly transmitting the end-alert signal comprises:
transmitting a device identification code;
receiving an open session command;
transmitting device data including data related to the event; and
receiving a close session command.

12. A method according to claim 11, wherein the device identification code further comprises a medical event uplink code.

13. A method according to claim 11, further comprising adjusting the status of a successful session bit of the IMD to inform the IMD that the wireless transmission of the alert signal was successful.

14. A method according to claim 1, wherein wirelessly transmitting an end-alert signal comprises:
transmitting a device identification code;
receiving an open session command;
transmitting device data including at least one of: a data set related to the end of the event, a temporal event-length, an event-type code; and
receiving a close-session command.

15. A method according to claim 1, wherein wirelessly transmitting an alert signal comprises:
transmitting a device identification code;
determining whether the device identification code was received, by monitoring for an open session command;
retrying the wireless transmission if the device identification code was not received;
transmitting device data if the device identification code was received;
determining whether the device data was received by monitoring for a close-session command;
retrying the wireless transmission if the device data was not received.

16. A method according to claim 15, wherein retrying the wireless transmission occurs after a delay.

17. A method according to claim 1, wherein wirelessly transmitting the end-alert signal comprises:
transmitting a device identification code;
determining whether the device identification code was received by monitoring for an open session command;
retrying the wireless transmission if the device identification code was not received;
transmitting device data if the device identification code was received;
determining whether the device data was received by monitoring for a close-session command;
retrying the wireless transmission if the device data was not received.

18. A method according to claim 17, wherein retrying the wireless transmission occurs after a delay.

19. A method according to claim 1, wherein the event relates to pulmonary edema excursion.

20. A method according to claim 1, wherein the event relates to a blood pressure excursion.

21. A method according to claim 1, wherein the event relates to a glucose level excursion.

22. A method according to claim 1, wherein the event relates to a neurological event.

23. A method according to claim 1, wherein the event relates to a low drug level in a drug infusion device.

24. A method according to claim 1, wherein the event relates to physiologic electrolyte concentrations.

25. A method according to claim 1, wherein the event relates to one of a protein level and a cardiac arrhythmia.

26. An improved alert system for alerting a care giver to an occurrence of an event detected by an implantable medical device, the alert system comprising:
means associated with the implantable medical device for detecting the occurrence and termination of a device- and/or physiologic-related event;
means associated with the implantable medical device for transmitting an alert signal after detection of the occurrence of the event and for transmitting an end-alert signal after detection of the termination of the event;
means for receiving the alert signal and for receiving the end-alert signal; and
means for communicating that the event has occurred, after receiving the alert signal, and for communicating that the event has terminated, after receiving the end-alert signal.

27. An alert system according to claim 26, further comprising a patient alert system for alerting the patient to the occurrence of the event.

28. An alert system according to claim 27, wherein the patient alert system is configured to begin alerting the patient to the occurrence of the event when the occurrence of the event is detected, and stops alerting the patient to the occurrence of the event when the end of the event is detected.

29. An alert system according to claim 27, wherein the patient alert system comprises a part of the implantable medical device.

30. An alert system according to claim 29, wherein the patient alert system comprises a viewable portion of a monitor.

31. An alert system according to claim 30, wherein the portion of the monitor comprises an LED that is turned on to indicate the occurrence of the event, and is turned off to indicate that the event has terminated.

32. An alert system according to claim 26, wherein the means for receiving the alert signal and for receiving the end alert signal comprises a monitor.

33. An alert system according to claim 26, wherein the means for receiving the alert signal and for receiving the end alert signal comprises a communications link transceiver.

34. An alert system according to claim 26, wherein the means for communicating to the care giver that the event has occurred and for communicating to the care giver that the event has terminated comprises a patient management network.

35. An alert system according to claim 26, wherein the means associated with the implantable medical device for detecting the occurrence and termination of the event comprises a thoracic impedance sensor.

36. An implantable medical device comprising:
a control processor for detecting a termination of a previously detected event and providing an end-alert signal; and
a telemetry system to wirelessly transmit the end-alert signal subsequent to the termination of the event.

37. An implantable medical device according to claim 36, further comprising detecting an initial occurrence of the event by determining when a measured value goes outside a normal range.

38. An implantable medical device according to claim 37, wherein detecting the termination of the event comprises determining when the measured value returns to within the normal range.

39. An implantable medical device according to claim 37, wherein the normal range is user selectable.

40. An implantable medical device according to claim 39, wherein the normal range is dynamically defined.

41. An implantable medical device according to claim 37, further comprising memory controlled by the control processor, the memory comprising a successful session bit to allow the implantable medical device to determine whether the wireless transmission of the alert signal has been successfully communicated.

42. A computer readable medium for storing instructions for a method operated under computer processor control, said method comprising an improved method of providing clinician- and/or patient-alerts indicating a termination of an event relating to a patient, wherein a prior detected occurrence of an event by an implantable medical device (IMD) wirelessly transmits an alert signal from the IMD indicating the occurrence of the event, said medium comprising:
instructions for detecting the termination of a previously detected event with an IMD; and
instructions for wirelessly transmitting an end-alert signal from the IMD indicating the termination of the event.

43. A medium according to claim 42, further comprising:
instructions for receiving the end-alert signal with a monitor;
instructions for transferring the end-alert signal from the monitor to a patient management network; and
instructions for communicating information regarding the event that occurred and terminated to at least one of a care giver, a hospital, a clinic, a clinician, the patient, said information comprising at least one of:
an event duration, an event type, a time the event started, a time the event ended, an aggregate event burden, a device intervention code, an activity sensor input for the patient.

44. A medium according to claim 42, further comprising:
instructions for receiving the end-alert signal with a monitor;
instructions for transferring the end-alert signal from the monitor to a patient management network; and
instructions for communicating that the event has terminated to at least one of a care giver, a hospital, a clinic, a clinician, the patient.
